(19) United States
(12) Patent Application Publication
(10) Pub. No.: US 2008/0243134 A1
(43) Pub. Date: Oct. 2, 2008

(54) BIDIRECTIONALLY RATCHETING SURGICAL INSTRUMENT

(76) Inventors: Kurt P. Limberg, Milwaukee, WI (US); Douglas A. Moyer, Racine, WI (US)

Correspondence Address: QUARLES & BRADY LLP, 411 E. WISCONSIN AVENUE, SUITE 2040, MILWAUKEE, WI, 53202-4497, US

(21) Appl. No.: 11/693,053

(22) Filed: Mar. 29, 2007

(51) Int. Cl. A61B 17/58

(52) U.S. Cl. 606/104; 192/43.1; 81/62

(57) ABSTRACT

A tool for driving a workpiece during surgical procedures has a handle. A gear with teeth thereon is rotatably supported along an axis within the handle. Two pawls are pivotally supported by the handle adjacent the gear and are orbital about the axis upon rotation of the handle. Each pawl has teeth that engage the gear teeth to transmit torque from the handle to the workpiece. Two springs yieldingly urge the pawls into engagement the gear teeth. A cap, with an actuator thereon, is rotatable with respect to handle. The cap has a first position in which the actuator retracts the first pawl out of engagement with the gear teeth, a second position in which the actuator retracts the second pawl out of engagement with the gear teeth, and a third position in which neither the first pawl nor the second pawl is retracted by the actuator.

### CROSS-REFERENCE TO RELATED APPLICATIONS

Not Applicable

### STATEMENT REGARDING FEDERALLY SPONSORED RESEARCH OR DEVELOPMENT

Not Applicable

### BACKGROUND OF THE INVENTION

1. Field of the Invention

The present invention relates to ratcheting surgical instruments, such as those for fastening screws or bolts, and more particularly, to such instruments which can be set to ratchet in either of two rotational directions or locked into a non-ratcheting state.

2. Description of the Related Art

Ratcheting drivers are commonly employed to apply fasteners, such as screws, bolts and the like. One type of ratcheting driver has a handle shaped like that of a conventional fixed blade screwdriver. However, the working end of the handle contains an actuator that maneuvers a gear and pawl assembly to selectively enable the handle to rotate the gear in either direction while allowing ratcheting in the opposite direction. A tool bit, such as a screwdriver blade or fastener socket, is attached to the gear and is driven by the handle motion.

In using a ratcheting driver, torque is applied by the user's hand to the handle and then via the pawl to the gear. The torque is transferred further to the tool bit which engages a workpiece, such as a screw, nut, or bolt. It is important to have the assembly arranged for optimum transmission of the applied hand torque in a reliable and consistent manner. That achievement is dependent on the construction, mounting, and location of the pawls.

Ratcheting drivers of this type are used as surgical instrument for orthopedic operations and other procedures. For example, a surgeon uses a ratcheting instrument to insert screws into bones to fasten plates to the skeleton of the patient. Surgical instruments have special requirements that common tools do not have to meet. Such instruments must be capable of withstanding high sterilizing temperatures of an autoclave. Any lubricants must be approved by the Food and Drug Administration of the United States Government if the surgical instrument is to be used in that country. Ratcheting driver also must be very durable as its failure during surgery may have serious consequences.

### SUMMARY OF THE INVENTION

A ratcheting surgical instrument has a handle to which a ratchet housing is fixedly attached. A gear is rotatably located within the housing and a tool bit is operably connected to the gear. A first pawl is pivotally disposed in the housing and selectively engages the gear to enable the handle to drive the gear and the tool bit only in a first direction. A second pawl also is pivotally disposed within the housing and selectively engages the gear to enable the handle to drive the gear and the tool bit only in a second direction. In a preferred embodiment of the surgical instrument, each pawl has a plurality of teeth that mesh with teeth of the gear to reliably transfer of torque between those components. Preferably, separate compression springs bias the first and second pawls with respect to the housing and toward engagement with the gear.

An actuator selectively retracts the first pawl away from engagement with the gear and retracts the second pawl away from engagement with the gear. In a preferred embodiment, the actuator is attached to a cap that can be rotated into different positions with respect to the housing. In a first position of the cap, actuator retracts the first pawl out of engagement with the teeth of the gear, in a second position the actuator retracts the second pawl out of engagement with the teeth of the gear, and in a third position neither the first pawl nor the second pawl is retracted by the actuator.

In the preferred construction of the surgical instrument, the housing is secured to a metal core and the handle is formed by a plastic, preferably an elastomer and in particular silicone rubber, that is molded around the core and the housing. In addition, the tool bit has a shaft that extends through a bore in the gear and the shaft has an end section onto which a nut is threaded. At least a portion of the bore in the gear has a non-circular cross section and the tool bit has a segment that mates with that non-circular cross section to rotationally lock the tool bit to the gear.

### BRIEF DESCRIPTION OF THE DRAWINGS

FIG. 1 is a side view of a ratcheting surgical instrument according to the present invention;

FIG. 2 is a longitudinal cross sectional view along line 2-2 in FIG. 1;

FIG. 3 is an enlarged view of a drive assembly in FIG. 2;

FIG. 4 is an exploded view of the surgical instrument;

FIG. 5 is an isometric view of a cap of the surgical instrument;

FIG. 6 is a cross sectional view along line 6-6 in FIG. 1; and

FIG. 7 is a longitudinal cross sectional view along line 7-7 in FIG. 1.

### DETAILED DESCRIPTION OF THE INVENTION

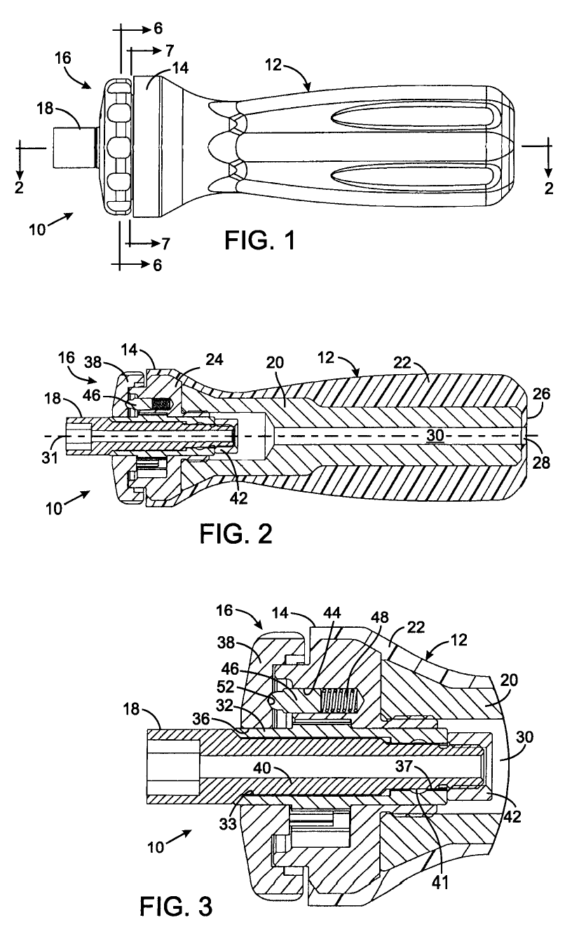

With initial reference to FIG. 1, a ratcheting surgical instrument 10 has a handle 12 with a working end 14 to which a gear and pawl assembly 16 is attached. A tool bit 18 is inserted into the gear and pawl assembly and may comprise a socket for a nut or bolt, a blade to engage a screw, or other type of tool for engaging a workpiece.

As shown in FIG. 2, the handle 12 comprises a tubular metal core 20 and an outer grip 22 of plastic, that preferably is an elastomer and in particular silicone rubber. A ratchet housing 24 also is part of the handle 12 and is threaded into an open end of the core 20 and extends partially outward from the working end 14 of the handle. The ratchet housing 24 and core 20 are assembled and placed within a mold, so that the plastic grip 22 then is over-molded around the core and housing. This secures the interior handle components within the grip 22. The outer surface of the core 20 is not round or at least that surface has depressions into which the grip material penetrates during molding to lock the core and grip together. Therefore when torque is applied to the handle 12, the grip 22 cannot rotate about the core 20.

The opposite end 26 of the grip 22 that is remote from the working end 14 has an aperture 28 there through that opens into the central bore 30 extending along an axis 31 through the core 20. This aperture 28 enables a guide wire and the like to pass through the surgical instrument 10 to the work site in the patient.

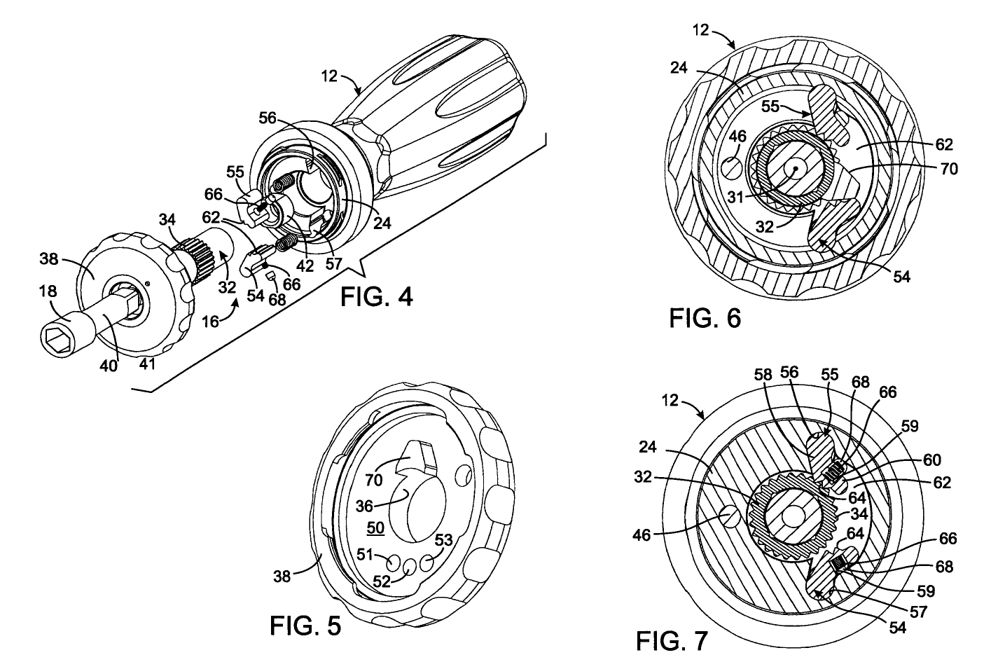

The remaining components are then assembled onto the molded handle 12. With reference to FIGS. 3 and 4, a gear 32 is inserted through the central aperture of the housing 24 and extends into an enlarged portion of the bore 30 in the core 20. The gear 32 is a tube with a center section having a plurality of teeth 34 that project outwardly from the exterior circumferential surface. One end of the gear 32 fits through a center aperture 36 in a cap 38. The cap 38 is rotatably attached to the open end of the housing 24 being interlocked thereon. The cap is able to rotate on the housing partially around the smooth end portion of the gear 32. The tool bit 18 has a shaft 40 extending into a central bore 33 in the gear 32 and projects out of the interior end of the gear. A section of the tool bit shaft 40 near its interior end has a square cross section 41 that mates with a similarly shaped cross section 37 of the bore in the gear 32 and shown in FIG. 3. That mating rotationally locks the gear onto the shaft 40. A nut 42 is threaded onto the interior end of the tool bit shaft 40 and bonded thereto by a commercially available thread locking adhesive, thereby securing the tool bit 18 to the gear and pawl assembly 16.

With reference to FIGS. 3 and 6, the housing 24 has a blind aperture 44 within which a detent 46, in the form of a plunger, is slideably received and biased outwardly therefrom a helical compression spring 48. The exterior end of the detent 46 has a hemispherical surface that successively nests into three hemispherical depressions 51, 52 and 53 in the interior surface of the cap 38, which depressions are shown in FIG. 5. As will be described, the detent 46 slides into one of these depressions 51-53 in each of three different rotational positions of the cap 38 about the end of the handle 12. Such engagement releasably holds the cap in the respective rotational position which places the ratcheting gear and pawl assembly 16 into one of three operating states, as will be described.

The gear and pawl assembly 16 further includes a pair of oppositely arranged pawls 54 and 55 that are received within grooves 56 and 57, respectively, within the housing 24. Each pawl 54 and 55 has a dog-leg shape with a rounded first end 58 that is pivotally received within the curved groove 56 or 57 in the housing. An arm 60 projects from the curved end 58 and out of the groove 56 into a cavity 62 within the housing 24. Each pawl has a plurality of teeth 64 that selectively engage the teeth 34 of the gear 32. The plurality of teeth 64 on each pawl 54 and 55 provide a secure engagement over a relatively large surface area between the respective pawl and the gear 32. Each pawl has an aperture 59 in its outward facing surface within which one end of a pawl spring 66 is received. The other end of the pawl spring 66 is received within a cap-like, hemispherical plunger 68 that engages a wall of the housing cavity 62. The pawl spring 66 biases the respective pawl 54 or 55 toward engagement with the teeth 34 of gear 32. As seen in FIG. 5, a pawl actuator 70 project from the interior surface 50 of the cap 38 and into the housing cavity 62 as illustrated in FIG. 6. The pawl actuator 70 has a generally truncated triangular shape.

In a first rotational position of the cap 38 shown in FIG. 6, the pawl actuator 70 engages and pivots the first pawl 54 away from engagement with the teeth 34 of the gear 32. In a second rotational position of the cap 38 the pawl actuator 70 pushes the second pawl 55 away from the gear teeth 34. In a third cap position which is in between the first and second positions, the pawl actuator 70 does not engage either of the pawls 54 or 55 and therefor both pawls engage the teeth 34 of the gear 32 in response to the bias force of their respective pawl spring 66.

The user of the surgical instrument 10 selects the desired direction of ratchet motion by rotating the cap 38 about the handle 12 into one of the three detent positions. The first position at one extreme of the cap rotation shown in FIGS. 6 and 7 is used to loosen a fastener, such as a bolt, that is held by the tool bit 18. In this cap position, the pawl actuator 70 pushes the first pawl 54 away from engagement with the gear 32, so that only the second pawl 55 now engages the teeth 34 of that gear. When the user applies torque to the handle 12 in the clockwise direction FIGS. 6 and 7, the handle provides additional force that causes the teeth 64 second pawl 55 into mesh with the gear teeth 34 and the second pawl cannot to ride out of engagement with the gear 32. Thus application of this torque to the handle 12 rotates the gear 32 and the tool bit 18 secured thereto. However in this state, rotation of the handle in the counterclockwise direction in FIG. 6, allows the second pawl 55 to pivot within the housing 24 so that its teeth ride into and out of the teeth of the gear 32. Now the pawls 54 and 55 orbit about the axis of the handle without applying torque to the gear and tool bit 18. Thus rotation of the handle in this direction allows ratcheting of the surgical instrument 10 and the gear 32 and the tool bit 18 do not drive the fastener.

The hemispherical plunger 68 enables an end of the pawl spring 66 to pivot with respect to the wall of the housing cavity 62 as the associated pawl 54 or 55 ratchets over the gear teeth 34. This avoids the pawl spring from binding or bending during the ratcheting which could impede the operation of the surgical instrument 10. The spring pivoting enabled by the plunger 68 also facilitates the retraction and engagement of each pawl 54 and 55 with respect to the gear 32 in response to rotation of the cap 38.

To tighten a fastener, the cap 38 placed into the second position, which is at the opposite end of the cap rotation with respect to the first position shown in FIGS. 6 and 7. In this position, the pawl actuator 70 retracts the second pawl 55 away from the gear 32. At the same time, the first pawl 54 is forced into engagement with the gear 32 by its pawl spring 66. Thus the state of the gear and pawl assembly 16 is now reversed so that the handle 12 can be rotated in a direction that tightens the fastener engaged by the tool bit 18. In other words, when counterclockwise torque is applied to the handle 12, the first pawl 54 transfers that torque to the gear 32 and the tool bit 18. In this state the teeth 64 of the first pawl 54 are unable to move out of engagement with the gear teeth 34. However, when clockwise torque is applied to the handle 12 in the orientation of components in FIG. 6, the first pawl 54 pivots within its housing groove 57 slipping over the teeth of gear 32 thereby allowing free rotation of the handle without producing movement of the tool bit 18.

When the cap 38 is rotated by the user into the center, or third position, the pawl actuator 70 is located between the two pawls 54 and 55 without engaging either pawl. Thus both the first and second pawls 54 and 55 are biased by their respective springs 66 into engagement with the teeth of the gear 32. In this dual pawl engaging position, the first pawl 54 prevents the handle 12 from ratcheting when rotated in the counterclockwise direction and the second pawl 55 prevents the handle from ratcheting when rotated in the clockwise direction. Thus, the handle is now locked to the gear in both rotational directions.

The foregoing description was primarily directed to a preferred embodiment of the invention. Although some attention was given to various alternatives within the scope of the invention, it is anticipated that one skilled in the art will likely realize additional alternatives that are now apparent from disclosure of embodiments of the invention. Accordingly, the scope of the invention should be determined from the following claims and not limited by the above disclosure.

1. A surgical instrument having a ratchet mechanism comprising:
a handle;
a housing fixedly attached to the handle;
a gear rotatably disposed within the housing;
a tool bit operably connected to the gear within the housing;
a first pawl pivotally disposed within the housing and selectively engageable with the gear to enable the handle to drive the gear and the tool bit only in a first direction;
a first compression spring biasing the first pawl with respect to the housing and toward engagement with the gear;
a second pawl pivotally disposed within the housing and selectively engageable with the gear to enable the handle to drive the gear and the tool bit only in a second direction;
a second compression spring biasing the second pawl with respect to the housing and toward engagement with the gear; and
an actuator that selectively retracts the first pawl away from engagement with the gear and selectively retracts the second pawl away from engagement with the gear.

2. The surgical instrument as recited in claim 1 further comprising:
a first pawl plunger abutting the housing and within which an end of the first spring is received; and
a second pawl plunger abutting the housing and within which an end of the second spring is received.

3. The surgical instrument as recited in claim 1 wherein when the first pawl away is retracted from engagement with the gear, the first spring is located entirely in an aperture in the first pawl; and when the second pawl away is retracted from engagement with the gear, the second spring is located entirely in an aperture in the second pawl.

4. The surgical instrument as recited in claim 1 wherein each of the first pawl and the second pawl has a plurality of teeth that mesh with teeth of the gear.

5. The surgical instrument as recited in claim 1 further comprising a metal core to which the housing is secured and wherein the handle is formed by plastic that is molded around the core and the housing.

6. The surgical instrument as recited in claim 1 wherein the tool bit has a shaft that extends through a bore in the gear and the shaft has an end section onto which a nut is threaded.

7. The surgical instrument as recited in claim 1 wherein the gear has a bore with at least a portion having a non-circular cross section, and the tool bit has a segment that mates with non-circular cross section of the gear to rotationally lock the tool bit to the gear.

8. The surgical instrument as recited in claim 1 wherein the actuator has a first position in which the first pawl is retracted, a second position in which the second pawl is retracted, and a third the actuator in which neither the first pawl nor the second pawl.

9. The surgical instrument as recited in claim 1 wherein the actuator is attached to a cap that is rotatable on the housing.

10. The surgical instrument as recited in claim 9 further comprising a detent connected to the housing and biased away there from; and the cap has a plurality of depressions into which the detent is received in different rotational positions of the cap with respect to the housing.

11. A surgical instrument having a ratchet mechanism comprising:
a handle;
a housing fixedly attached to the handle;
a gear rotatably disposed within the housing and having a first plurality of teeth;
a tool bit operably connected to the gear within the housing;
a first pawl pivotally disposed within the housing and having a second plurality of teeth that selectively engage first plurality of teeth to enable the handle to drive the gear and the tool bit only in a first direction;
a second pawl pivotally disposed within the housing and having a third plurality of teeth that selectively engage first plurality of teeth to enable the handle to drive the gear and the tool bit only in a second direction; and
an actuator for selectively retracting the first pawl away from engagement with the gear and retracting the second pawl away from engagement with the gear.

12. The surgical instrument as recited in claim 11 further comprising:
a first spring biasing the first pawl with respect to the housing and toward engagement with the gear; and
a second spring biasing the second pawl with respect to the housing and toward engagement with the gear.

13. The surgical instrument as recited in claim 11 wherein the first and second spring are compression springs.

14. The surgical instrument as recited in claim 11 further comprising a metal core to which the housing is secured and wherein the handle is formed by plastic that is molded around the core and the housing.

15. The surgical instrument as recited in claim 11 wherein the tool bit has a shaft that extends through a bore in the gear and the shaft has an end section onto which a nut is threaded.

16. The surgical instrument as recited in claim 11 wherein the gear has a bore with at least a portion having a non-circular cross section, and the tool bit has a segment that mates with non-circular cross section of the gear to rotationally lock the tool bit to the gear.

17. The surgical instrument as recited in claim 11 wherein the actuator has a first position in which the first pawl is retracted, a second position in which the second pawl is retracted, and a third the actuator in which neither the first pawl nor the second pawl is retracted.

18. The surgical instrument as recited in claim 11 wherein the actuator is attached to a cap that is rotatable on the housing.

19. The surgical instrument as recited in claim 18 further comprising a detent held in the housing and biased away there from; and the cap has a plurality of depressions into which the detent is received in different rotational positions of the cap with respect to the housing.

20. The surgical instrument as recited in claim 11 further comprising a cap rotatable about the housing and to which the actuator is attached, wherein the cap has a first position in which the actuator retracts the first pawl, a second position in which the actuator retracts the second pawl, and a third position in which the actuator retracts neither the first pawl nor the second pawl.

21. A surgical instrument, for rotationally driving a workpiece, comprising:
a handle having an axis and a hollow interior;
a gear with teeth thereon and being rotatably supported along the axis in the handle hollow interior for rotationally driving the workpiece;
two pawls pivotally supported by the handle adjacent the gear and being orbital about the axis upon rotation of the handle and with each of the pawls having teeth engageable with the gear teeth for transmitting torque from the handle to the workpiece;
two springs yieldingly urging the pawls into positions in which the teeth of each pawl engage the teeth of the gear; and
a cap rotatable with respect to handle and having an actuator thereon, wherein the cap has a first position in which the actuator retracts the first pawl out of engagement with the teeth of the gear, a second position in which the actuator retracts the second pawl out of engagement with the teeth of the gear, and a third position in which the actuator retracts neither the first pawl nor the second pawl.

22. The surgical instrument as recited in claim 21 wherein the two spring are compression springs.

23. The surgical instrument as recited in claim 21 further comprising a tool bit with a shaft that extends through a bore in the gear, and the shaft has an end section onto which a nut is threaded.

24. The surgical instrument as recited in claim 21 wherein the gear has a bore with at least a portion having a non-circular cross section; and further comprising a tool bit has a segment that mates with non-circular cross section of the gear to rotationally lock the tool bit to the gear.

25. The surgical instrument as recited in claim 21 further comprising a detent held in the handle and biased away there from; and the cap has a plurality of depressions into which the detent is received in different positions of the cap with respect to the handle.
